SEQUENCE LISTING (2 sequences)

SEQ ID NO: 1 - DNA, 25 bp, Homo sapiens
agctcaaagccatttctccgctgac

SEQ ID NO: 2 - DNA, 25 bp, Homo sapiens
ctagcaactggagaagccccatgcc

(19) United States
(12) Patent Application Publication
(10) Pub. No.: US 2012/0207681 A1
(43) Pub. Date: Aug. 16, 2012

(54) CHEMICAL COMPOSITIONS TO DETECT AND TREAT AMYLOID IN A PATIENTS BRAIN AND RETINA

(76) Inventor: Steven Verdooner, Granite Bay, CA (US)

(21) Appl. No.: 13/312,613

(22) Filed: Dec. 6, 2011

Related U.S. Application Data
(60) Provisional application No. 61/420,741, filed on Dec. 7, 2010.

(51) Int. Cl. A61K 49/00; A61P 25/28; A61P 35/04; A61P 27/02; A61P 9/10; A61K 31/12; A61P 25/00; B82Y 5/00

(52) U.S. Cl. 424/9.1; 514/679; 977/773; 977/700; 977/906; 977/927

(57) ABSTRACT

The present invention is a chemical composition to detect and treat amyloid in a patient's brain or a retina, that include a nanoparticle or a Nano biopolymer delivery platform to deliver any combination of gadolinium, one or more contrast agents, one or more therapeutics and curcumin to mark said amyloid and a polymalic acid scaffold.

This application claims priority to U.S. Provisional Application 61/420,741 filed on Dec. 7, 2010, the entire disclosure of which is incorporated by reference.

### TECHNICAL FIELD & BACKGROUND

Imaging of amyloid-beta plaques, including amyloid and amyloid-beta peptides and other pathology and anatomical features in the brain and retina is often unobtainable without the use of one or more specialized contrast agents and an expensive PET scanner and other equipment.

It is an object of the invention to provide a chemical composition that is utilized in a variety of configurations that include standard MRI imaging devices in combination with a dual-labeled nanoparticle or Nano biopolymer delivery platform for gadolinium and/or other contrast/binding agents and curcumin.

It is an object of the invention to provide a chemical composition that provides traditional brain amyloid imaging that relies upon one or more contrast agents that are difficult to manage in combination with distinct and relatively expensive PET scan equipment exclusively found in a select number of medical facilities.

It is an object of the invention to provide a chemical composition that is a combination of nanoparticle or Nano biopolymer technology in combination with one or more standard MRI (PET or optical) imaging devices which are much more accessible to patients.

What is needed is a chemical composition that is utilized in a variety of configurations that include standard MRI imaging devices in combination with a dual-labeled nanoparticle or Nano biopolymer delivery platform for gadolinium and/or other contrast/binding agents and curcumin that provides traditional brain amyloid imaging that relies upon one or more contrast agents that are difficult to manage in combination with distinct and relatively expensive PET scan equipment exclusively found in a select number of medical facilities and that is a combination of nanoparticle or Nano biopolymer technology in combination with one or more standard MRI, PET or one or more other optical imaging devices which are much more accessible to patients.

### BRIEF DESCRIPTION OF THE DRAWINGS

The present invention will be described by way of exemplary embodiments, but not limitations, illustrated in the accompanying drawing in which like references denote similar elements, and in which:

FIG. 1 illustrates a front view of a chemical composition to detect and treat amyloid in a patient's brain and retina, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

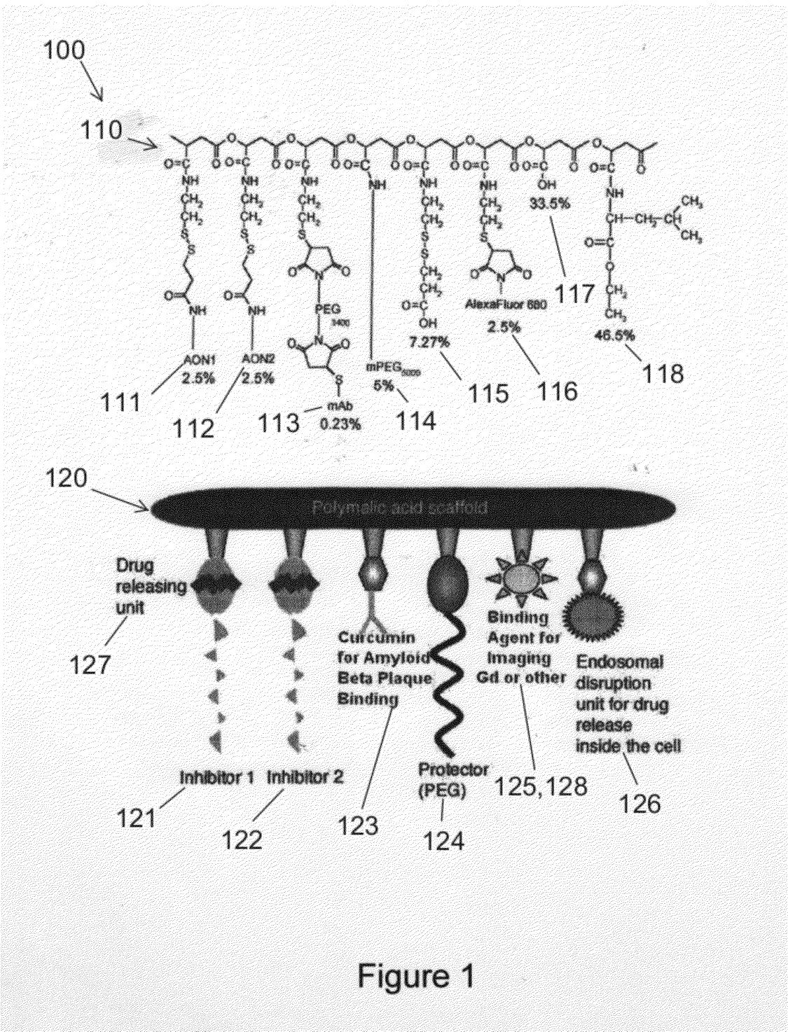

FIG. 1 illustrates a front view of a chemical composition 100 to detect and treat amyloid in a patient's brain and retina, in accordance with one embodiment of the present invention.

The chemical composition 100 includes a platform 110 and a polymalic acid scaffold 120. The platform 110 consists of 2.5% AON1 111, 2.5% AON2 112, 0.23% mouse anti-human TfR mAb 113, 5% mPEG 114, 7.27% L-leucine ethyl ester 115, 2.5% AlexaFluor 680 C2-maleimide 116, 33.5% poly (β-L-malic acid) (PMLA) 117 and curcumin 118. The polymalic acid scaffold 120 consists of a first inhibitor 121, a second inhibitor 122, curcumin 123, a protector PEG 124, a binding agent 125 and an endosomal disruption unit 126. Both 2.5% AON1 111, 2.5% AON2 112 have a drug releasing unit 127. The curcumin 123 in the polymalic acid scaffold 120 is utilized in amyloid beta plague binding. The binding agent 125 is for imaging Gd or other imaging agent 128 and the endosomal disruption unit 126 is to release the chemical composition 100 within a cell.

The chemical composition can be used to detect amyloid in a person's brain and retina utilizing a variety of imaging techniques including MRI, PET and optical imaging of curcumin-labeled plus a contrast agent such as gadolinium delivered via a nanoparticle or Nano biopolymer delivery system. Furthermore, the labeled cells may be visualized via fluorescent microscopy after tissue biopsy. These nanoparticles or Nano biopolymers could be used to track cells in vivo. This basic platform that contains curcumin (which is used to bind to amyloid plaques) may be modified with different fluorophores, targeting and therapeutic agents for diagnosing, studying and treating a variety of conditions including but not limited to Alzheimer's disease, traumatic brain injury, metastasis cell, stem cell, and or immune cell trafficking, macular degeneration and other ocular disorders among other suitable applications.

The chemical composition can utilize a variety of imaging techniques to image amyloid in the brain and or retina including MRI, PET and/or optical suitable imaging techniques or any combinations thereof. Curcumin may be combined with gadolinium and/or other contrast/binding agents and is delivered to plaques whereby curcumin binds to amyloid beta plaques and is subsequently imaged.

The chemical composition involve utilizing a variety of imaging techniques to image amyloid beta plaques in the brain and or retina including but not limited to MRI, PET, optical coherence tomography (OCT) and other optical imaging techniques. Curcumin is combined with gadolinium and/or other contrast/binding agents and is delivered via a biodegradable nontoxic Nano platform delivery system to plaques whereby curcumin binds to amyloid beta plaques for targeting purposes.

The chemical composition involves utilizing a variety of imaging techniques to image amyloid beta plaques in the brain and or retina including but not limited to, MRI, PET, OCT, and optical imaging techniques. Curcumin is combined with gadolinium and/or other contrast/binding agents and is delivered via a Nano biopolymer, Polycefin delivery system that delivers curcumin and a contrast agent (gadolinium and/or others) to plaques whereby curcumin binds to amyloid beta plaques for targeting.

The chemical composition involves utilizing a variety of imaging techniques to image amyloid beta plaques in the brain and or retina including, but not limited to, MRI, PET, OCT and optical imaging techniques. Curcumin is combined with gadolinium and/or other contrast/binding agents, and also combined with therapeutic agents and is delivered via a new Nano biopolymer, Polycefin delivery system that delivers curcumin and a contrast agent (gadolinium and/or others) to plaques where by curcumin binds to amyloid beta plaques.

The chemical composition involves utilizing a variety of imaging techniques to image amyloid beta plaques in the brain and or retina including, but not limited to MRI, PET, OCT and optical imaging techniques. Curcumin is combined with gadolinium and/or other contrast/binding agents, therapeutic agents, and is delivered via a biodegradable non-toxic Nano platform delivery system to plaques where by curcumin binds to amyloid beta plaques.

The chemical composition involves utilizing a variety of imaging techniques to image amyloid beta plaques in the brain and or retina including, but not limited to MRI, PET, OCT and optical imaging techniques. Curcumin is combined with gadolinium and/or other contrast/binding agents, and is delivered via a biodegradable non-toxic Nano platform delivery system to plaques where by curcumin binds to amyloid beta plaques.

The chemical composition utilizes curcumin as the therapeutic agent for removal of amyloid in the brain and/or retina for the treatment of (including but not limited to) Alzheimer's disease, macular degeneration, traumatic brain injury and other disorders. The curcumin is combined with a biodegradable nontoxic Nano platform delivery system delivered to the eye for the treatment of macular degeneration and other ocular disorders. Delivery includes (but is not limited to) intravitreal injection, subconjunctival injection, systemic IV injection, systemic IV injection coupled with photo-activation and topical eye drops. The curcumin serves as the binding or targeting agent to amyloid in the retina. The curcumin is utilized for removal of amyloid in the brain and/or retina for the treatment of (including but not limited to) Alzheimer's disease, macular degeneration, traumatic brain injury and other suitable disorders.

The chemical composition involves utilizing a Nano biopolymer, Polycefin delivery system that delivers therapeutics and/or contrast agents to the eye via (including, but not limited to) intravitreal injection, subconjunctival injection, systemic IV injection, systemic IV injection coupled with photo-activation, topical eye drops, for the treatment of macular degeneration, diabetic retinopathy, dry eye, and other ocular disorders.

The chemical composition is constructed from lipid monomers with diacetylene bonds that are sonicated and photolyzed to form polymerized nanoparticles where a plurality of cells is efficiently labeled with these nanoparticles.

The chemical composition involves utilizing a variety of imaging techniques to image amyloid beta plaques in the brain and or retina including (but not limited to) MRI, PET, OCT and other suitable optical imaging techniques. Curcumin is combined with gadolinium and/or other contrast/binding agents, and/or also combined with therapeutic agents and is delivered via a new platform for conjugate synthesis, poly (β-L-malic acid) (PMLA) delivery system that delivers curcumin and a contrast and or therapeutic agent to plaques whereby curcumin binds to amyloid beta plaques in the brain or retina.

The chemical composition and production of Poly (β-L-malic acid) combined with curcumin. Poly (β-L-malic acid) (PMLA) is harvested from the culture broth of P. polycephalum M3CVII (ATCC 204388) microplasmodia, grown for 4 days at 25° C. with the addition of 30 g/L D-glucose and 30 g/L CaC03 (pH 5.5). When the pH is dropped to 4.8, fermentation is terminated by shifts to a pH of 7.5 and 15° C. After clearing by centrifugation (500×g), the supernatant is diluted with one part of 0.05 M Tris-HCI pH 7.5 and pumped at 10 L/h from bottom to top of a 1.5 L column (12 em diameter, capacity for 10 L of broth) containing Streamline DEAE cellulose, then washed with 20 mM Tris-HCI buffer (pH 7.5) containing 0.2 M NaCI to remove the yellow pigment, and eluted with 20 mM Tris-HCI buffer containing 0.7 M NaCI from top to bottom. The PMLA containing fraction is adjusted to 0.1 M CaC12 and poly (malate) Ca2+-salt precipitated with 70-80% (v/v) ethanol at −20° C. The ice-cold ethanol-washed precipitate is dissolved in a minimum volume of distilled water and PMLA fractionated over Sephadex G25 into portions of 60-90 kDa, 50-60 kDa, and 20-50 kDa (Mw by HPLC-SEC with polystyrene sulfonate as standard). The calcium salt was converted into the acid by passage over Amperlite 120 H+, freeze-dried, and stored at −80° C. The obtained polymer is white powder, soluble in acetone, showing crystallization during solvent removal under vacuum. It is highly pure with regard to elementary analysis, HPLC-SEC analysis, thin layer chromatography (TLC)/ninhydrin reaction, optical rotation, 200-300 nm wavelength absorbance, infrared absorbance, C13/H1 NMR spectra), and devoid of protein, nucleic acids, polysaccharide, as well as UV-absorbing low molecular compounds [8-12]. PMLA in the 18 L culture broth amounted to 20-25 g. The yield after purification was 50-60%. This yield is combined with curcumin and or imaging contrast/binding agents, and/or therapeutic agents for the treatment of (including but not limited) Alzheimer's disease, macular degeneration, traumatic brain injury, and other disorders, where curcumin serves as a binding and targeting agent for amyloid in the brain and/or retina.

The polycefin structure version used contains mouse anti-human TfR mAb (RVS 10) instead of mouse anti-rat TfR mAb (OX-26) in the original version. AlexaFluor 680 C2-maleimide from Molecular Probes-Invitrogen (Carlsbad, Calif.) was employed as a fluorescent reporter. Custom made Morpholino™ (phosphorodiamidate Morpholino oligomer) AON1 has sequence listing 001 and AON 2 has sequence listing 002 (Gene Tools, Inc., St. Louis, Mo.) for human laminin-8 a4 and -1 chains that are listed after FIG. 1.

Mouse monoclonal anti-human TfR antibody RVS10 (Chemicon International, Temecula, Calif.) is used in this Polycefin variant, which also contained L-leucine ethyl ester instead of previously used valine. The synthesis from poly (β-L-malic acid) (Mw 50,000; Mw/Mn 1.3) followed the protocol previously described.

The polymalic acid scaffold is combined with Gd or other contrast agent, and potential therapeutic agents together with curcumin used for amyloid beta plaque binding. The composition can also include one or more nonspherical structures that include tubular structures such as mesoporous silica Nano rods that are loaded with gadolinium (or other contrast agents), therapeutic agents, and curcumin in the pores, carbon nanotubes with iron oxide nanoparticle handles fused to one end, zeolite Nano containers with combinations of dye and lanthanides bound to the surface or introduced into channels and necklace-like cables of metal-poly (vinyl alcohol) loaded with iron oxide nanoparticles. The multimodal nanoparticle agents can be constructed by embedding probes of various functionalities within an inert matrix. This allows mixing of imaging agents of predetermined performance characteristics, with only minimal interference by the surrounding matrix.

The chemistry for assembling the carrier matrix is generally much less complicated than those described earlier for synthesizing magnetic or luminescent nanoparticles. In addition, the proportion of imaging probes of different types can be controlled simply by varying stoichiometry of starting reagents. The carrier matrix can be assembled as a plurality of nanoparticles first then swelled to infuse imaging probes into the matrix, or probes that can be covalently attached to one or more matrix components prior to particle formation. These multimodal nanoparticle agents consist of curcumin, contrast agents such as Gd, and/or therapeutic agents for targeted delivery to amyloid beta plaques.

The curcumin is combined with Gd and/or other imaging contrast agents, and/or therapeutic agents via nanoparticle carriers that include polymer, silica, or one or more dendrimers. The curcumin and the binding I contrast agent (Gd and/or other) and/or therapeutic agents can also be synthesized via macromolecular carriers or small molecule multimodal probes. As previously described, curcumin is utilized as a therapeutic agent for the treatment of macular degeneration, diabetic retinopathy, and other ocular disorders and is delivered via (but not limited to) intravitreal injection, subconjunctival injection, systemic IV injection, systemic IV injection coupled with photo-activation, topical eye drops, oral delivery, and or in combination with Polycefin and/or other biodegradable non-toxic Nano platform delivery system, or nanoparticle delivery system.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A chemical composition to detect and treat amyloid in a patient's brain or a retina, comprising:
a nanoparticle or a nano biopolymer delivery platform to deliver any combination of gadolinium, one or more contrast agents, one or more therapeutics and curcumin to mark said amyloid, and
a polymalic acid scaffold.

2. The composition according to claim 1, wherein said curcumin binds to a plurality of amyloid beta plaques and is used in combination with said contrast agents to allow targeted imaging and delivery of said one or more therapeutics.

3. The composition according to claim 2, wherein said curcumin binds to said amyloid beta plaques and in combination with said gadolinium and allows direct imaging of said amyloid beta plaques.

4. The composition according to claim 2, wherein said curcumin is combined with gadolinium, one or more contrast agents or said one or more therapeutics to be delivered via conjugate synthesis and a poly (β-L-malic acid) delivery system.

5. The composition according to claim 4, wherein said curcumin is combined with said delivered poly (β-L-malic acid).

6. The composition according to claim 1, wherein said curcumin is delivered to said patient via said nanoparticle or said Nano biopolymer delivery platform.

7. The composition according to claim 6, wherein said nanoparticle or said Nano biopolymer delivery platform is used to track said amyloid.

8. The composition according to claim 7, wherein said nanoparticle or said Nano biopolymer delivery platform is used to track said amyloid in vivo.

9. The composition according to claim 1, wherein said curcumin is bound via a biodegradable and non-toxic delivery Nano platform able to specifically carry said curcumin and one or more other drugs to said amyloid beta plaques.

10. The composition according to claim 9, wherein said curcumin is bound via said Nano biopolymer and said biodegradable and non-toxic delivery Nano platform able to specifically carry said curcumin and one or more other drugs to said amyloid beta plaques.

11. The composition according to claim 10, wherein said curcumin is combined with said non-toxic delivery Nano platform delivered to said patient selected from the group consisting of intravitreal injection, subconjunctival injection, systemic IV injection, systemic IV injection coupled with photo-activation or topical eye drops.

12. The composition according to claim 11, wherein said curcumin serves as a binding agent or a target agent to indicate said amyloid in said patient's retina.

13. The composition according to claim 11, wherein said curcumin serves as a therapeutic agent.

14. The composition according to claim 1, wherein said curcumin is combined with said gadolinium, said one or more contrast agents and via one or more nanoparticle carriers that include a polymer, a silica, or one or more dendrimers.

15. The composition according to claim 1, wherein said curcumin is combined with said gadolinium, said one or more contrast agents and is synthesized via one or more macromolecular carriers or one or more small molecule multimodal probes.

16. The composition according to claim 1, wherein said Nano biopolymer delivers to said therapeutics to said patient's eye selected from the group consisting intravitreal injection, subconjunctival injection, systemic IV injection, systemic IV injection coupled with photo-activation or topical eye drops.

17. The composition according to claim 1, wherein said polymalic acid scaffold is combined with said gadolinium, said one or more contrast agents and said curcumin to bind said amyloid.

18. The composition according to claim 1, wherein said polymalic acid scaffold is a polycefin.

19. The composition according to claim 1, wherein said composition includes a plurality of multimodal nanoparticle agents that are constructed by a plurality of embedding probes within an inert matrix.

20. The composition according to claim 1, wherein said composition is utilized to treat Alzheimer's disease, a traumatic brain injury, a plurality of metastasis cells, a plurality of stem cell, immune cell trafficking and macular degeneration and one or more ocular and one or more brain disorders.

21. The composition according to claim 1, wherein said composition treats macular degeneration, diabetic retinopathy and one or more ocular diseases.

22. The composition according to claim 21, wherein said nanoparticle or said nano biopolymer delivery platform delivers any combination of one or more said contrast agents and said one or more therapeutics to said patient's eye selected from the group of intravitreal injection, subconjunctival injection, oral consumption, systemic IV injection, systemic IV injection coupled with photo-activation or topical eye drops.
